(19) United States
(12) Patent Application Publication
(10) Pub. No.: US 2024/0285278 A1
(43) Pub. Date: Aug. 29, 2024

(54) APPARATUS AND METHOD FOR USE IN MINIMALLY INVASIVE ANASTOMOSIS CREATION AND/OR ENDOLUMINAL NAVIGATION

(71) Applicant: BariaTek Medical, Paris (FR)

(72) Inventors: Christophe BASTID, Marseille (FR); Thierry MANOS, Marseille (FR); Youssef BIADILLAH, Maisons-Laffitte (FR)

(73) Assignee: BariaTek Medical, Paris (FR)

(21) Appl. No.: 18/572,565

(22) PCT Filed: Jun. 22, 2022
(86) PCT No.: PCT/EP2022/066977

§ 371 Date: Dec. 20, 2023

(30) Foreign Application Priority Data
Jun. 22, 2021 (EP) 21315100.4

(51) Int. Cl. A61B 17/11; A61B 17/00; A61B 17/12; A61B 17/30

(52) U.S. Cl. CPC A61B 17/1114; A61B 17/12045; A61B 17/12136; A61B 2017/00292; A61B 2017/1103; A61B 2017/306

(57) ABSTRACT

Apparatus (10) insertable endoluminally into a body duct, for example the gastro-intestinal tract, and operable to manipulate body duct tissue to create a target site for an anastomosis by bringing sections of the body duct tissue into close relation with each other, the apparatus comprising:
an anchor assembly (12) including an anchor for anchoring at a selected position within the body duct;
at least a first pulling assembly (14a, 14b) configured for contracting a portion of the body duct in a lengthwise direction from within the body duct, by pulling distant tissue towards the anchor assembly;
a lateral movement device (22, 24) configured for moving first and second sections of the body duct tissue that are spaced lengthwise along the body duct, laterally into wall-to-wall engagement with each other; and
a deployment device for deploying one or more tools and/or devices for creating an anastomosis.

### FIELD OF THE INVENTION

The present invention relates to the fields of (i) anastomosis creation using minimally invasive techniques, and (ii) endoluminal navigation. Some embodiments relate to endoluminal apparatus for use in anastomosis creation.

### BACKGROUND TO THE INVENTION

An anastomosis is a surgical cross-connection or bridge between two different sections of body duct lumen. The gastro-intestinal tract is the luminal route in the body from the esophagus to the anus. Anastomoses formed somewhere along or in the gastrointestinal tract are one form of therapy used to treat digestion-related problems, such as diabetes, obesity, bowel diseases and obstructions. An anastomosis can be used to bypass a portion of the gastro-intestinal tract, such as a portion of the small intestine, to avoid sensitive areas or to influence or reduce absorption of nutrients.

Currently, open-surgery provides most comprehensive access to the internal anatomy for forming an anastomosis. However, open-surgery is highly invasive, and unsuitable for many patients and conditions to be treated. Minimally invasive procedures have been proposed, but significant challenges remain in forming anastomoses equally effectively by a minimally invasively procedure, especially endoluminally. In an endoluminal procedure, one or more tools are introduced into the body principally through the body duct in which the anastomosis is to be made.

Current endoluminal techniques are best suited to anastomosis procedures that are relatively shallow in the body duct and formed between wall sections that are already situated naturally in proximity to each other. This limits, for example, the versatility of the procedure for the small intestine which, in most adults, can have a length of up to 6 or 7 meters and is folded on itself many times in the abdomen.

Current techniques for endoluminal navigation within the gastrointestinal tract also rely on the ease of following the tract within the anatomy, and ease of passage from one section of the tract to another. However, unless complex and expensive steering is implemented, there are regions of the anatomy, where the tendency of an apparatus to naturally follow a minimum curvature path, may impede ease of navigation.

### SUMMARY OF THE INVENTION

It would be desirable to address and/or mitigate one or more of the above issues.

Aspects of the invention are defined in the claims.

Broadly speaking, one aspect of the present invention provides apparatus insertable into a body duct (for example, the gastrointestinal tract) and operable to manipulate body duct tissue to create a target site for an anastomosis by bringing sections of the body duct tissue into close relation with each other.

The apparatus can comprise an anchor assembly and at least a first pulling assembly.

The anchor assembly includes an anchor for anchoring at a selected position within the body duct.

The first pulling assembly is configured for contracting a portion of the body duct in a lengthwise direction from within the body duct, by pulling distant tissue towards the anchor assembly. Contraction of the tissue in a lengthwise direction may also be referred to herein as foreshortening.

In some embodiments, the first pulling assembly comprises at least one elongate member that is extendable and retractable for movement within the body duct, and at least one distal balloon inflatable for frictionally engaging an interior surface of the body duct to enable the frictionally engaged tissue to be pulled towards the anchor assembly.

Such an arrangement can greatly facilitate manipulation of body duct tissue, enabling a tissue wall section that is naturally distant from the anchor to be brought much closer to the anchor assembly by pulling the tissue atraumatically from within the body duct itself. The body duct is contracted in the lengthwise direction, effectively concertinaing, or gathering up, the body wall tissue to pull the distant tissue closer. Optionally, the apparatus is configured for endoluminal introduction into the body duct.

In some embodiments, the elongate member may be extendable from the anchor assembly within the body duct for accessing the distant tissue. The balloon may be expanded for frictionally engaging tissue at the distant position. The elongate member may be retracted back towards the anchor assembly to pull the frictionally engaged tissue towards the anchor assembly, and thereby contract the body duct lengthwise to bring distant tissue closer to the anchor assembly.

In some embodiments, the first pulling assembly optionally comprises first and second elongate members, each carrying a respective (first and second, respectively) inflatable balloon.

This can permit stepwise contraction of the body duct tissue and/or is especially suitable for tortuous body ducts, such as the small intestine. For example, in use, the first elongate member may be extended with its balloon deflated, while the second elongate member remains retracted, optionally with its balloon inflated to hold back any pre-contracted tissue. Once the first elongate member has been extended to access distant tissue, the first balloon may be inflated to engage against and anchor the distant tissue. The second balloon may be deflated, and the second elongate member extended adjacent to the first elongate member and balloon, and the second balloon inflated to engage against tissue adjacent to the first balloon. The first and second members may be retracted in unison, with at least one balloon, optionally both balloons, frictionally engaging body duct tissue to enable the tissue to be pulled towards the anchor assembly during the retraction. Thereafter, the first balloon may be deflated, and the first member again extended to access a next region of distant tissue, while the second balloon keeps the contracted body duct tissue in its contracted state. The process can be repeated multiple times in order to contract the body duct tissue in lengthwise steps, and hence facilitate access to distant and folded tissue even deep within, for example, the small intestine.

In some embodiments, the first and second elongate members of the first pulling assembly may be nested one within another. At least one, optionally both, of the elongate members may be tubular. For example, the first elongate member may be nested within, and slidable lengthwise with respect to, the second elongate member.

At least one elongate member of the first pulling assembly may be at least partly deflectable and/or steerable, for example at its tip, to facilitate navigation when extending within the body duct.

The term “balloon” is used herein to refer to any inflatable body or inflatable cuff, that may be distended by an inflation fluid. The inflation fluid may be liquid (e.g. saline) or it may be gas (e.g. air). At least one, optionally each, balloon may be generally spherical in shape (e.g. generally frusto-spherical), or generally egg-shaped, or generally cylindrical in shape. Any other shape of balloon may be used as desired to suit the body duct.

The anchor of the anchor assembly may be deployable from a collapsed condition to a deployed condition. The deployable anchor may, for example, comprise an inflatable anchoring balloon.

One or more assemblies of the apparatus can comprise or consist of polyether ether ketone, polyethylene terephthalate, polyimide and/or polyamide.

Howsoever the first puling assembly and/or the anchor assembly is implemented, the apparatus may optionally further comprise a lateral movement device and/or a deployment device for deploying one or more tools and/or devices for creating an anastomosis.

The lateral movement device, if provided, may be configured for moving first and second sections of the body duct tissue that are spaced lengthwise along the body duct, laterally towards and/or into wall-to-wall engagement with each other.

The lateral movement device can enable two sections of body duct tissue, for example, previously distant sections brought closer together by the first pulling assembly, to be (e.g. further) pulled towards and/or into wall-to-wall engagement with each other to become suitable as a target site for an anastomosis. For example, if the two wall sections are close but offset from one another, the second pulling assembly can pull the two sections into wall-to-wall engagement, such that exterior surfaces of the wall sections are in engagement. The lateral movement device may, for example, cause the body duct to bend such that the wall sections approach each other.

By using the first pulling assembly first to contract the body duct lengthwise, and hence draw distant tissues closer together, the lateral movement device need move tissue only a relatively short distance in order to bring tissues towards or into wall-to-wall engagement.

The combination of the first pulling assembly and the lateral movement device can enable access to and manipulation of tissues even deep within the anatomy, increasing the versatility and efficacy of minimally invasive procedures for anastomosis creation, especially endoluminally.

Various types of lateral movement device are possible. For example, the lateral movement device could be implemented as part of the elongate member of the first pulling assembly, configured to allow lateral forces to be applied to move or bend the duct laterally. In some embodiments, the lateral movement device comprises a second pulling assembly configured for spanning positions along the body duct by passing outside the body duct, and for pulling the spanned positions towards wall-to-wall engagement with each other, e.g. exterior wall-to-wall engagement.

Similarly to that which is already described above, by using the first pulling assembly first to contract the body duct lengthwise, and hence draw distant tissues closer together, the second pulling assembly need span only a relatively short distance in order to connect and pull tissues into wall-to-wall engagement. Operation of the second spanning assembly to span across the tissue positions outside the duct needs only local guidance, for example, fluoroscopy or ultrasound imaging, because the distance is relatively short.

In some embodiments, the second pulling assembly comprises an extendable and retractable spanning element for passing through tissue walls at two different sections and spanning the distance between them. The second pulling assembly further comprises a connecting device in the form of a deployable distal shoulder for engaging behind one of the tissue wall sections to enable the tissue wall sections to be pulled towards wall-to-wall engagement with each other. Additionally or alternatively, a connecting device in the form of a suction port may be used to allow the spanning element to connect externally to a tissue wall using negative pressure to maintain the connection.

In some embodiments, the spanning element is configured for penetrating through the tissue wall(s) when extended. Optionally, the spanning element is at least partly deflectable and/or steerable, for example, at its tip, for navigating when extending to span the distance between the first and second sections.

In some embodiments, the spanning element may be extendable from the anchor assembly and be advanceable to pass through the adjacent tissue wall and outside the body duct. The spanning element may be advanceable towards a distal end of the first pulling assembly, to penetrate back through the tissue wall near or at the first pulling assembly.

Alternatively, the spanning element may be extendable from the first pulling assembly and be advanceable to pass through the adjacent tissue wall and outside the body duct. The spanning element may be advanceable towards the anchor assembly, to penetrate back through the tissue wall near or at the anchor assembly.

In either case, the distal shoulder may be deployed to anchor behind the penetrated tissue wall, thereby permitting the tissue wall to be pulled when the spanning element is retracted.

The deployable shoulder may, for example, comprise an inflatable balloon or a deployable mechanical structure, such as a self-expanding frame or stent.

A partially deployed stent could comprise a deployable shoulder which in an at least partially deployed state can be used for retracting a body duct section and in the fully deployed state serves as an implanted stent for creating an anastomosis. The stent optionally creates a temporary through-channel anastomosis.

A temporary through-channel can allow digestive products to pass through the gastro-intestinal tract until a permanent anastomosis is created, thereby making a second operational procedure obsolete.

Additionally or alternatively to the second pulling assembly, the deployment device may be configured for deploying one or more tools and/or devices for creating an anastomosis. For example, the deployment device may be configured for deploying the one or more tools and/or devices at a position established by the second pulling assembly where the sections of duct wall are pulled towards wall-to-wall engagement.

Devices for creating an anastomosis may optionally include a stent for creating a stented-anastomosis, and/or a magnetic device for creating a magnetic compression anastomosis.

A stent for creating an anastomosis can be configured to be monolithic (a single fully connected piece) so that the stent is deployed on either sides inside the body duct being pierced through the body duct walls. The magnetic device may be configured to apply forces from both sides of adjacent tissue walls, or the device may be one component of a pair of separate devices deployable at different positions in the duct in register with one another, and attracted to each other by magnetic attraction. The other component may also be deliverable and deployable by the same apparatus (e.g. by a second deployment device of the apparatus).

Tools for creating an anastomosis may optionally include a tissue cutter and/or a tissue fixation device for inserting sutures and/or staples or other tissue fixings for joining the tissues of opposed duct walls sections together around mutual openings defining an anastomosis channel.

The device and/or tool for creating an anastomosis may be introducible through a working channel of the apparatus, and/or may be loadable or loaded into an accommodation region of the deployment device, the device and/or tool being deployable from the accommodation region upon operation of the deployment device.

In some embodiments the deployment device is positioned in proximal direction from the anchoring assembly. In an alternative embodiment the deployment device is positioned in distal direction from either the first advanceable balloon and/or second advanceable balloon. This arrangement allows deploying a tool for creating an anastomosis outside of the region of contracted body duct between the anchoring assembly and an advanceable balloon.

In some embodiments the apparatus comprises a connecting device, which can comprise a second pulling device.

The connecting device can be configured to bring the outer surfaces of the two sections of a body duct in close contact with each other at the target site for creating an anastomosis by bending the body duct and fixing the target site of the outer surface sections relative to each other.

The connecting device can comprise a suction device for aligning and/or positioning the body duct walls relative to each other and/or deploying a tissue cutter by applying a negative pressure.

In one embodiment at least a section of the apparatus, preferably the inner assembly can assume a bent configuration without piercing the body duct, thereby bringing the target site for the anastomosis of the two sections of the body duct into closer contact.

The anchoring assembly and/or the balloons can be conceived to assume a predetermined shape designed at an angle such that the contracted portion of the body duct assumes an arc which brings the sections of the body duct into closer contact. In one embodiment the anchoring assembly and/or balloons are configured to be non-symmetric around the inner assembly or outer assembly. The anchoring assembly and/or balloons can be configured to have a longer extension in the longitudinal direction on one side of the inner assembly, pulling assembly and/or outer assembly than on the other side in a deployed and/or inflated state such that the apparatus can assume a bent shape.

The tissue cutter can comprise a cutting element, a puncturing element, an electrical or optical ablation element. The tissue cutter can further comprise a cutting element which comprises a laterally expandable and/or collapsible profile and/or periphery. In one embodiment the cutting element comprises a counter surface which can be deployed on the opposing side of the body duct to be cut. The counter surface covers the target area of the body duct wall and thereby prevents leakage. In one embodiment of the apparatus, the counter surface also comprises a laterally expandable and/or collapsible profile and/or periphery.

The laterally expanded profile and/or periphery of the cutting element and/or the counter surface can preferably assume a predetermined dimension.

The cutting element further can be configured to be rotatable along its circumferential direction, optionally in a helical movement along the longitudinal direction.

The apparatus can comprise temporary compression elements for creating a compression anastomosis. In one embodiment the temporary compression elements comprise magnetic elements. The temporary compression elements are preferably positionable on either side of the body duct tissue at the target site to create a compression anastomosis.

The connecting device can comprise a spanning element.

The deployable shoulder can reversibly be converted to a stowed configuration and an expanded configuration. The deployable shoulder can comprise a balloon element, a shape-memory alloy, a wire mesh, a monolithic structure, a frame structure and/or a polygon structure such that the spanning element can increase its lateral dimension in the expanded configuration to a greater lateral dimension than the stowed configuration.

The increased lateral dimension by the deployable shoulder in the expanded configuration is preferably greater than an incision, puncture and/or cut by the tissue cutter.

The increase in lateral dimension is greater than 200 percent, optionally greater than 300 percent, in particular optionally greater than 400 percent.

A reversibly convertible balloon and/or assembly and/or element by unfolding to a predetermined lateral dimension allows for use of inelastic materials which are more resilient in view of wear and tear and thus increase safety of a patient.

The unfoldable balloon and/or assembly and/or element can ensure a fixed predetermined maximum and minimum lateral dimension when applying proper pressure and can frictionally engage the tissue to be pulled with maximised tightness.

In such an embodiment, different geometric shapes such as a folded zigzag structure and/or concertina structure could be conceived.

Such an unfoldable balloon and/or assembly and/or element does not rely on elastic deformation, so the material used can be radially stiff.

An unfoldable balloon and/or assembly and/or element may comprise or consist of polyamide, polyethylene terephthalate and/or polyether block amide.

In one embodiment the apparatus can comprise a pressuring device, optionally located in a region at a proximal end of the apparatus that can be brought into fluid communication with at least one balloon such that fluid is transferable from the pressuring device into the balloon, preferably into at least two balloons independently of each other. The pressuring device can reversibly convert the at least one balloon from the collapsed condition to an expanded condition.

The pressuring device can comprise a simple system of multiple syringes or a more sophisticated system of automated pumps.

It would also be conceivable that the pressuring device comprises a communication interface which automatically moves the apparatus without a user manually controlling the fluid supply. The user could enter an input into the communication interface, such as a length to be contracted of portion of the body duct.

It would also be conceivable that the pressuring device is located within the apparatus with a predetermined supply of compressed fluid.

The balloons are arranged in fluid communication with the pressuring device via at least one flow channel. In a preferred embodiment the balloons are in fluid communication with the pressuring device via individual flow channels. However, it is conceivable that fluid can be transferred from one balloon directly to another balloon with the pressuring device.

The apparatus can comprise at least one visualization element, preferably comprising at least one: (i) a camera, (ii) a radiopaque material, (iii) ultrasound impermeable material.

An optional visualization element may allow for locating the position of the delivery device.

The visualization element can preferably allow for locating the relative position of at least one balloon, preferably multiple balloons, relative to the anchoring assembly.

A further aspect of the invention, usable optionally independently of the preceding aspects or in combination with any of the preceding aspects, relates to endoluminal navigation within the gastro-intestinal tract.

This aspect provides apparatus for insertion into the gastrointestinal tract through a patient's mouth, and for navigating at the pylorus, the apparatus optionally according to any preceding aspect, the apparatus comprising:
a first elongate tubular member having a tip insertable into a patient's mouth an into the stomach antrum, the tube including a first region carrying an inflatable balloon configured such that inflation of the balloon causes the first region of the tube to displace away from the stomach wall; and
a second elongate member slidable within the first elongate tubular member, and translatable to extend from the tip of the first tubular member, and to pass through the pylorus from a position spaced away from the stomach wall by the balloon.

The shape of the anatomy in the region of the stomach antrum and the pylorus can cause problems for endoluminal navigation of devices, introduced via the patient's mouth, passing through the stomach towards the small intestine. By displacing the tip away from the stomach wall, for example, the stomach floor in the antrum, the tip can be better aligned with the pylorus in order to enable more straightforward and atraumatic navigation through the pylorus, for example, avoiding the tip lodging in the pyloric antrum.

The first portion may be near or at the tip of the first elongate tubular member.

In some embodiments, the apparatus is configured for delivering a device through the pylorus for placement at least partly within the duodenum and/or at least partly traversing the pylorus. Optionally, the device may comprise a duodenal liner sheath and/or an anchor for anchoring near or at the pylorus. The device may optionally be part of the apparatus.

A further aspect of the invention provides a method of endoscopically manipulating body duct tissue to create a target site for an anastomosis by bringing sections of body duct tissue into close relation with each other, using an apparatus insertable endoluminally into the body duct, the apparatus optionally as defined in any preceding aspect, the method comprising:
deploying an anchor of the apparatus to anchor the apparatus at a selected position within the body duct;
operating a first pulling assembly of the apparatus to contract a portion of the body duct in a lengthwise direction from within the body duct, by pulling distant tissue towards the anchor assembly;
the method further comprising at least one of the following steps:
operating a lateral movement device of the apparatus to move first and second sections of the body duct tissue that are spaced lengthwise along the body duct, laterally towards and/or into wall-to-wall engagement with each other; and/or
operating a deployment device to deploy one or more tools and/or devices for creating an anastomosis.

The step of operating the first pulling assembly may comprise contracting a portion of the body duct in a lengthwise direction such that the first and second sections of body duct tissue move from a distant relation to a closer relation in the lengthwise direction.

The optional step of operating the lateral movement device may further bring the first and second sections of body duct tissue from the closer relation into wall-to-wall engagement by relatively moving one or both of the first and second sections laterally with respect to the other.

A further aspect of the invention provides a method of endoscopically navigating within the gastro-intestinal tract of a patient, the method optionally including any of the method steps and/or apparatus of the any of the preceding aspects, the method comprising:
advancing a first elongate tubular member within the gastrointestinal tract to position a first portion of the first member in the region of a restriction of the tract to be traversed;
inflating a balloon of the first elongate tubular member to displace the first portion of the first elongate tubular member away from the wall of the tract; and
advancing a second elongate member within the first elongate member, to pass through the restriction of the tract from a position spaced from the tissue wall by the balloon of the first elongate tubular member.

The first portion of the first elongate member may be at or near a tip of the first elongate member.

In some embodiments, the restriction of the gastro-intestinal tract is a pylorus.

The method may optionally further comprise a step of deploying an implant into the gastro-intestinal tract. Optionally, the implant comprises at least one selected from: a duodenal sleeve; a duodenal anchor for a duodenal sleeve; a trans-pyloric anchor for a duodenal sleeve; a gastric anchor for a duodenal sleeve.

In any of the aspects disclosed herein, the apparatus or any component may optionally be formed by three-dimensional printing.

Although certain ideas, features and advantage have been highlighted above, protection is claimed for any novel feature disclosed herein and/or in the drawings, whether or not emphasis has been placed thereon.

### BRIEF DESCRIPTION OF THE DRAWINGS

FIG. 1 is a schematic side view of the distal region of an endoluminal apparatus

FIG. 2 is a schematic section illustrating introduction of the apparatus of FIG. 1 through the esophagus and stomach towards the small intestine.

FIG. 3 is a schematic section similar to FIG. 2 illustrating advancement of the apparatus of FIG. 1 to a selected position in the small intestine.

FIG. 4 is a schematic section similar to FIG. 3 illustrating anchoring of the apparatus of FIG. 1 at the selected position in the small intestine and a first phase of operation of a first pulling assembly of the apparatus to access distant tissue.

FIG. 5 is a schematic section similar to FIG. 4, illustrating a second phase of operation of the first pulling assembly of the apparatus to contract a region of the intestine lengthwise.

FIG. 6 is a schematic section similar to FIG. 5, illustrating a third phase of operation of the first pulling assembly to access distant tissue.

FIG. 7 is a schematic section similar to FIG. 6, illustrating a fourth phase of operation of the first pulling assembly.

FIG. 8 is a schematic section similar to FIG. 7, illustrating a fifth phase of operation of the first pulling assembly to further contract the intestine lengthwise.

FIG. 9 is a schematic section similar to FIG. 8, illustrating a first phase of operation of a lateral movement device.

FIG. 10 is a schematic section similar to FIG. 9, illustrating a second phase of operation of the lateral movement device.

FIG. 11 is a schematic section similar to FIG. 10, illustrating a third phase of operation of the lateral movement device.

FIG. 12 is a schematic section similar to FIG. 11, illustrating a fourth phase of operation of the lateral movement device.

FIG. 13 is a schematic section similar to FIG. 12, illustrating a deployment of an anastomosis device.

FIG. 14 is a schematic section similar to FIG. 13, illustrating the implanted anastomosis device after removal of the endoluminal apparatus.

FIGS. 15a-i are schematic sections illustrating in more detail, in isolation, operating steps of the apparatus of FIG. 1.

FIGS. 16a-f are schematic sections similar to FIG. 15 illustrating in more detail, in isolation, further operating steps of the apparatus of FIG. 1.

FIG. 17 is a schematic section illustrating introduction of a catheter towards the pylorus in a further example.

FIG. 18 is a schematic section illustrating deployment of a balloon in the pyloric antrum.

FIG. 19 is a schematic section illustrating traversal of the pylorus following deployment of the balloon.

### DETAILED DESCRIPTION OF PREFERRED EMBODIMENT

Non-limiting embodiments of the invention are now described by way of example only, with reference to the accompanying drawings. In the drawings, the same reference numerals are used to denote the same or equivalent features, whether or not described explicitly.

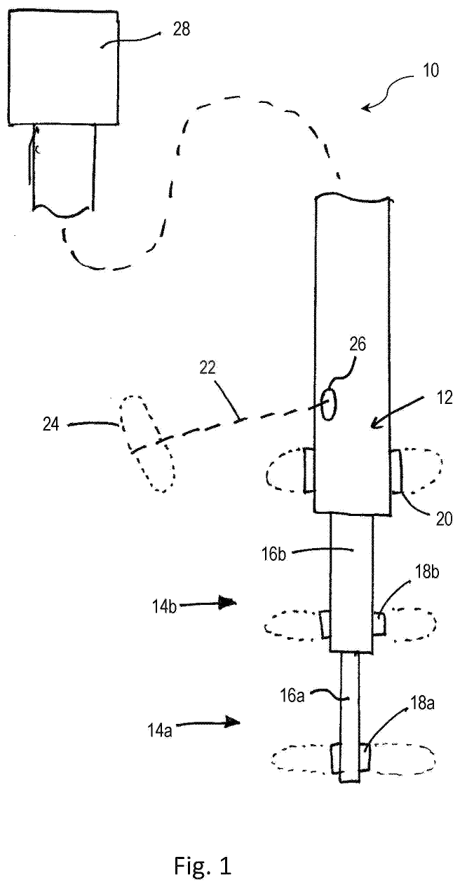

Referring to FIG. 1, endoluminal apparatus 10 is illustrated that is insertable into a body duct and operable to manipulate body duct tissue to create a target site for an anastomosis by bringing sections of the body duct tissue into close relation with each other. The apparatus 10 generally comprises at least an anchor assembly 12 and one or more inner assemblies 14a, 14b forming (e.g. collectively) a first pulling assembly. Two inner assemblies 14a and 14b are illustrated in this embodiment, although other embodiments could use only a single inner assembly 14, or three or more inner assemblies 14.

Each inner assembly 14a, 14b generally comprises a respective elongate member 16a, 16b carrying a respective distal balloon 18a, 18b (shown schematically in its deflated or collapsed state by a solid line, and in its inflated state by a broken line). The inner assemblies 14a, 14b are nested slidably one within another, the elongate members 16a, 16b being tubular. The innermost assembly 14a extends distally of its surrounding assembly 14b. Each assembly 14a, 14b is slidable at least partly independently of the other, and independently of the surrounding anchor assembly 12.

At least one of the inner assemblies (for example, at least the inner-most assembly 14a), and optionally both inner assemblies 14a and 14b is/are at least partly deflectable and/or steerable, for example, at their tips.

The anchor assembly 12 further comprises a deployable anchor 20, optionally an inflatable anchor balloon 20 shown schematically in its deflated or collapsed state by a solid line, and in its inflated state by a broken line.

The apparatus 10 further comprises a lateral movement device (illustrated later below), one part of which is a second pulling assembly formed collectively by a spanning element 22 with a deployable distal shoulder 24. The spanning element 22 is extendable and retractable with respect to the anchor assembly 12 via an aperture 26. The aperture 26 may optionally communicate with a working channel or accommodation region of the apparatus, described later. The spanning element 22 may be at least partly deflectable and/or steerable, for example, at its tip.

The deployable distal shoulder 24 may, for example, comprise an inflatable balloon, or it may comprise a deployable structure such as a self-expanding structure, e.g. a stent, carried by the spanning element 22.

The apparatus 10 optionally further comprises, at its proximal, end a handle and/or operator unit 28 by which the different elements described above may be manually or remotely controlled. For example, the unit 28 may comprise manual controls and/or ports for: manipulating the elongate members 16a and 16b; for controlling inflation of the balloons 18a and 18b collectively or independently; for controlling steering of steerable elements; for manipulating the spanning element 22; for controlling inflation of the balloons 20 and 24.

The balloons 20, 18a, 18b and 24 may have any suitable shape or shapes, for example selected from: spherical (or part spherical), doughnut-shaped, cuff-shaped, etc.

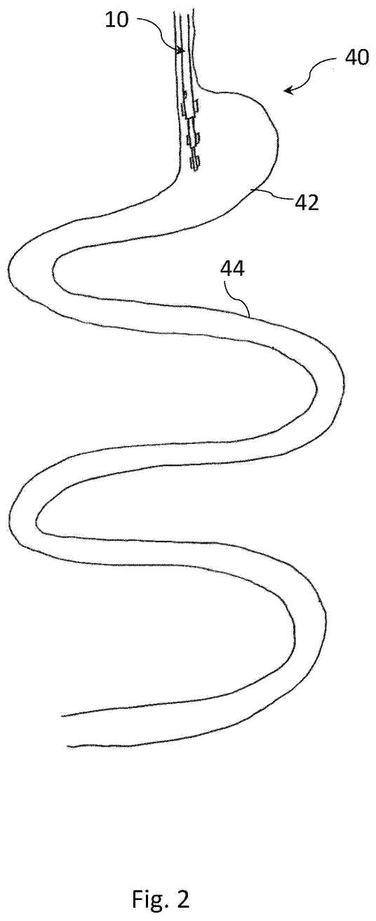

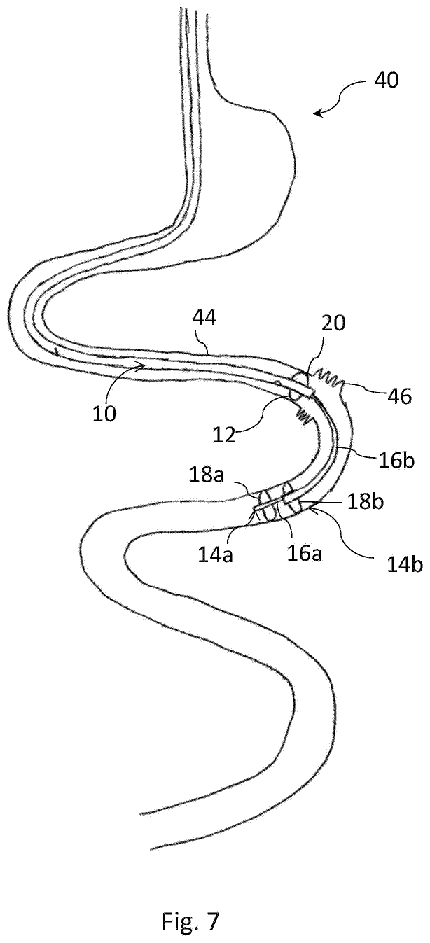

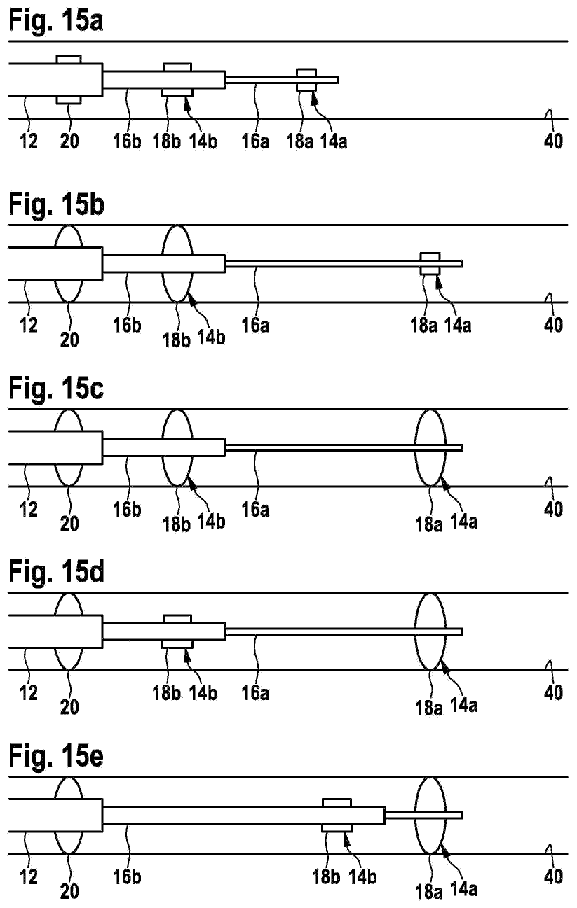

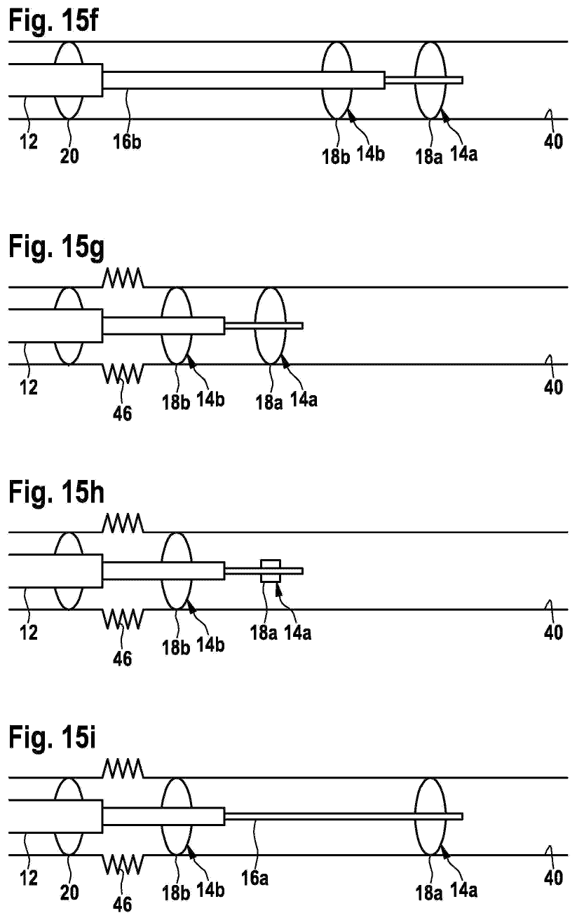

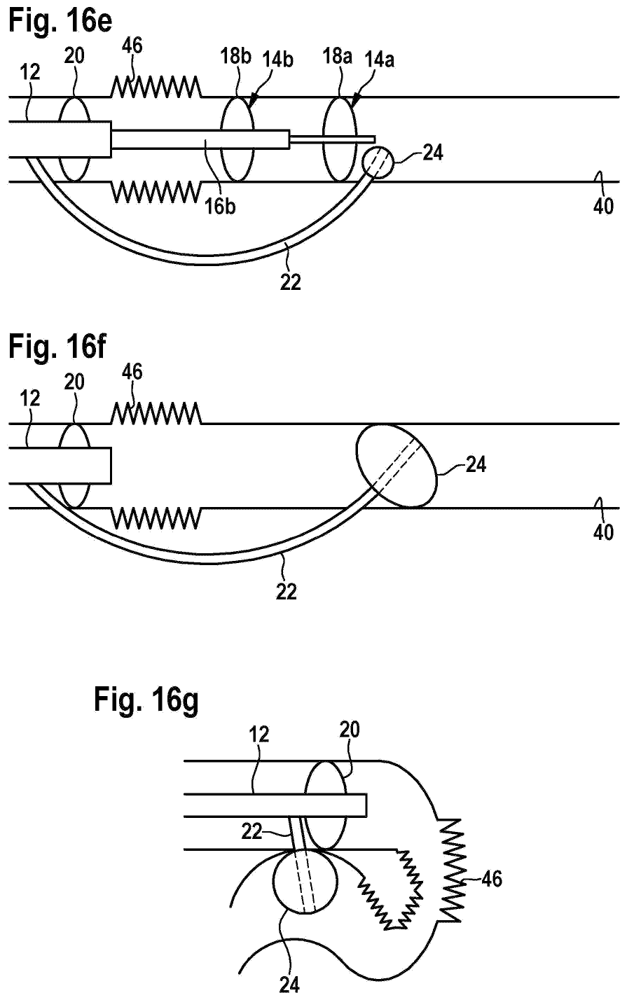

FIGS. 2-16 illustrate use of the apparatus 10 in a body duct 40 for endoluminally manipulating the body duct to create a target site for an anastomosis. FIGS. 2-14 illustrate the technique in a curved duct. FIGS. 15 and 16 illustrate operation steps more in isolation, so that more detail can be appreciated. FIGS. 15 and 16 may be for a straight duct, or FIGS. 15 and 16 may represent, in a straightened-out view, the body duct of FIGS. 2-14.

The same reference numerals are used in both groups of figures, and the same description applies universally.

Referring to FIGS. 2, 3 and 15a, the apparatus 10 is introducible endoluminally into the body duct in which it is desired to form an anastomosis. The apparatus 10 may be configured for introduction by placing the balloons 20, 18a, 18b and 24 in their deflated states, and retracting the inner assemblies 14a and 14b to their pulled-back positions, and completing retracting the spanning element 22 with respect to the anchor assembly 12. During advancement of the apparatus 10 in the body duct, and/or during advancement of either of the inner assemblies 14, the distal end may be steered or deflected from the handle 28 to facilitate navigation within the body duct.

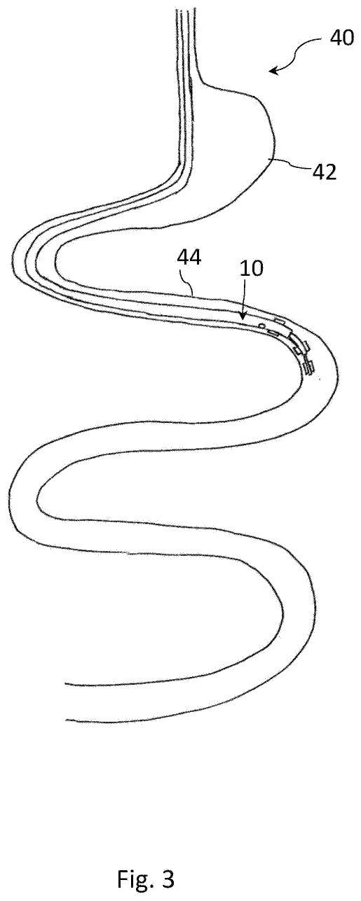

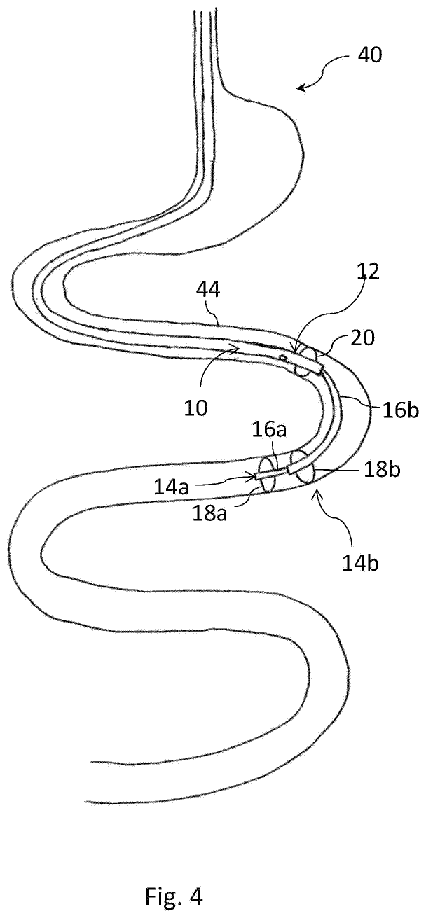

By way of example, the body duct may be the gastro-intestinal tract 40 of a patient. The apparatus 10 may be introducible through the mouth and esophagus, and advanced through the stomach 42 towards the pylorus and small intestine 44, until the distal end region of the apparatus reaches a selected position in the body duct (FIG. 3). For example, the apparatus may extend through the duodenum to a selected position within the jejunum. Referring to FIGS. 4 and 15b, the apparatus 10 is anchored at the selected position by inflating the anchor balloon 20 to engage surrounding body tissue.

FIGS. 4 to 8 and 15b to 16d illustrate operation of the first pulling assembly for contracting a portion of the body duct in a lengthwise direction from within the body duct, by pulling distant tissue towards the anchor assembly 12. Referring to FIGS. 4 and 15b, after anchoring at the selected position, at least the inner-most assembly 14a may be advanced distally (e.g. with the respective balloon 18a deflated) to access tissue that is distant relative to the anchor assembly 12, and the balloon 18a then inflated to frictionally engage the distant tissue.

Initially, prior to any contraction of the body duct, the other inner assembly 14 by may optionally be advanced in unison with the inner-most assembly 14a, as depicted in FIG. 4. Both balloons 18a and 18b may be inflated to frictionally engage the distant tissue. Alternatively, as depicted in FIGS. 15b to 15f, the assemblies 14a and 14b may be advanced individually one after the other. Referring to FIG. 15b, the inner assembly 14a is advanced first to access distant tissue, and the balloon 18a inflated (FIG. 15c) to frictionally engage the distant tissue. Referring to FIG. 15d, the inflatable balloon 18b of the other assembly 14b is deflated, and the assembly 14b advanced towards the distant tissue (FIG. 15e). Subsequently, the balloon 18b is inflated adjacent to the balloon 18a of the inner-most assembly 14a. This two-stage procedure may be preferred to facilitate easier navigation within a tortuous duct. The same two-stage procedure is also used as described below once some of the body duct tissue has been contracted lengthwise.

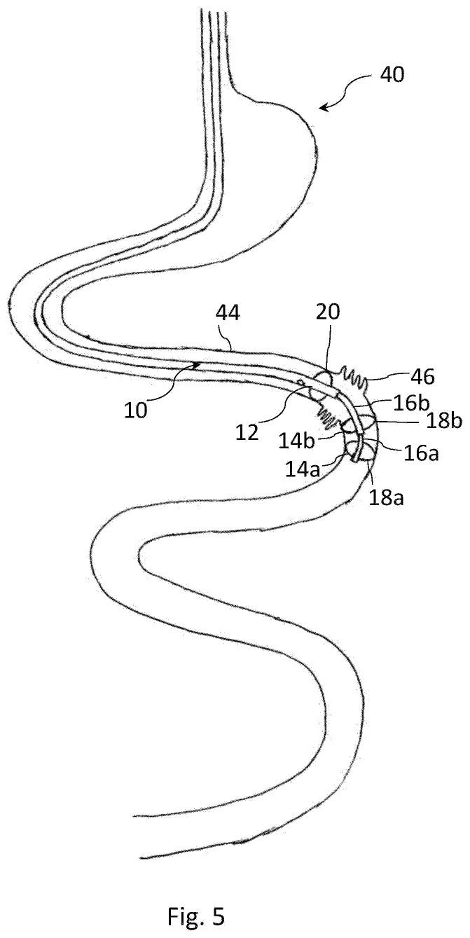

Referring to FIGS. 5 and 15g, retraction of the inner assemblies 14a and 14b pulls the frictionally engaged distant tissue towards the anchor assembly 12, thereby contracting or foreshortening the duct in the lengthwise direction. The wall tissue of the duct concertinas or collects near to the anchor assembly 12, as depicted at 46.

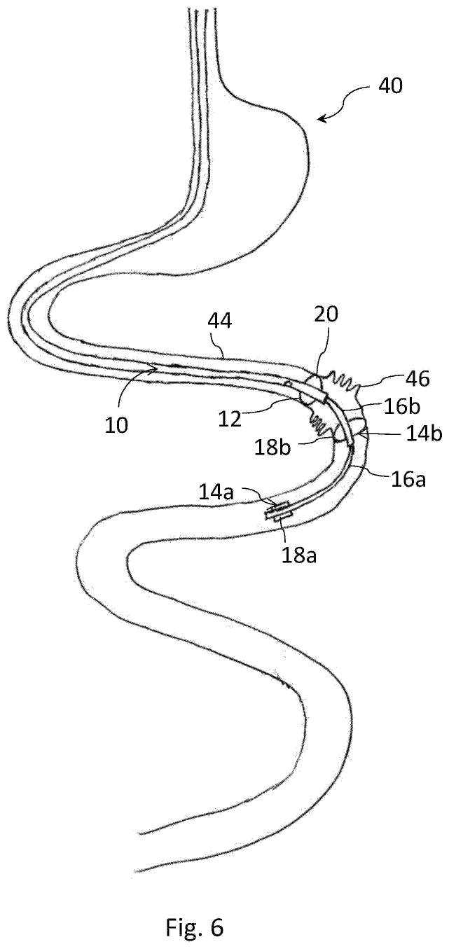

Referring to FIGS. 6 and 15h, while one inner assembly 14b is kept inflated to hold the contracted tissue in the contracted state, and overcome any tendency for the contracted tissue to rebound or re-expand lengthwise, the other (inner-most) assembly 14a is again extended to access a next region of distant tissue, with the balloon 18a in the deflated state. The elongate member 16a of the inner assembly 14a may be steered to assist in navigating movement within the body duct along a curved or tortuous path, and also avoid pulling against the contracted tissue during the distal advancement.

Once the distant tissue has been reached, the distal balloon 18a of the inner assembly 14a can again be inflated to frictionally engage the tissue (FIG. 15i). The balloon 18b of the other inner assembly 18b may then be deflated (FIG. 16a), and the elongate member 18b extended to approach the distant tissue (FIG. 16b). The balloon 18b may then be reinflated to engage the distant tissue next to the balloon 18a (FIG. 16c).

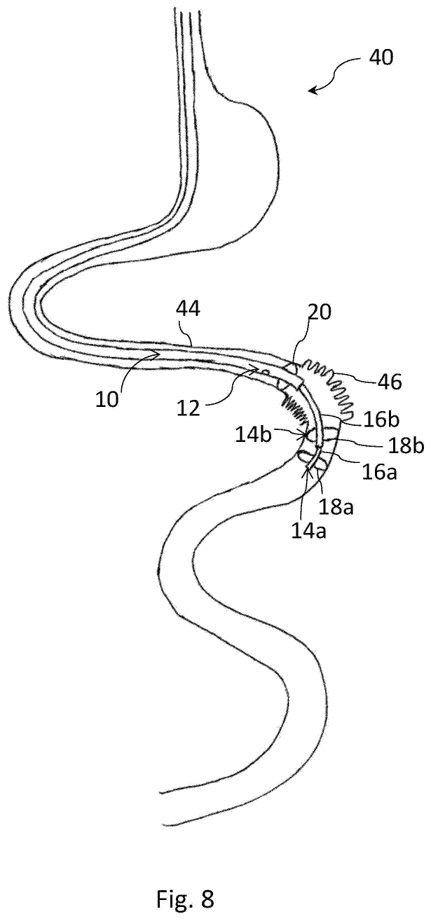

Referring to FIGS. 8 and 16d, retraction of the inner assemblies 14a and 14b, optionally in unison, pulls the frictionally engaged distant tissue towards the anchor assembly 12, such that the tissue adds to the concertinaed tissue collecting near the anchor assembly 12, thereby further contracting the duct lengthwise.

The above steps may be repeated as many times as appropriate to move distant tissue progressively towards the anchor assembly 12, for example, until a desired position along the duct has been reached, and/or a sufficient bypass length of body duct has been accumulated in the concertinaed tissue.

Provision of both balloons 18a and 18b assists in frictional engagement that is sufficiently firm to be able to pull tissue walls, yet with forces that are distributed and atraumatic for the tissue walls. Provision of both balloons 18a and 18b (and assemblies 14a and 14b generally) also enables a stepped approach to be used to pull distant tissue, with one balloon used to hold back pre-contracted tissue while the other balloon can be advanced (in its deflated state) to access other distant tissue. Provision of both balloons 18a and 18b (and assemblies 14a and 14b generally) also facilitates contraction of tissue that is not straight, by using a stepped approach. However, in other embodiments, a single inner assembly 14 and single balloon 18 could also be used instead.

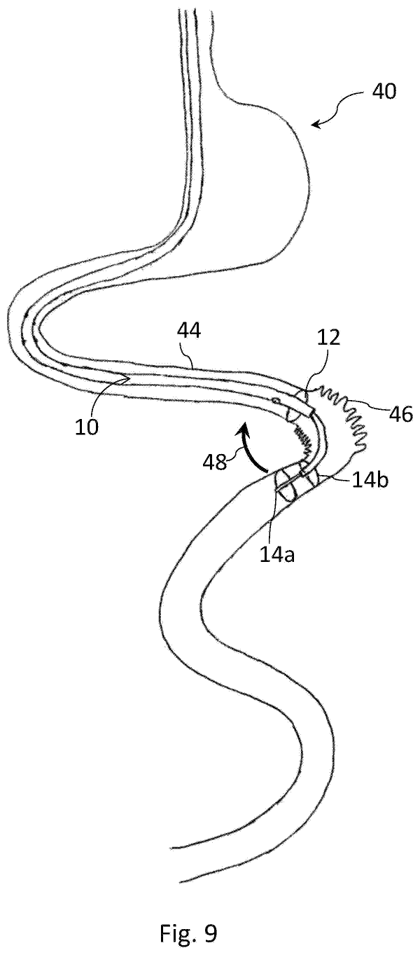

FIGS. 9 to 12 and 16e-g illustrate operation of one or more lateral movement devices to bring first and second sections of body duct tissue that are spaced apart from each other in the lengthwise direction of the duct, laterally towards and/or into wall-to-wall engagement with each other. Two different types of lateral movement device are described, which may each be used independently of the other, or both together in the same apparatus (as in the example of FIGS. 9 to 12).

Referring to FIG. 9, a first example of lateral movement device is illustrated in the form of a bending mechanism in one or both of the inner assemblies 14a, 14b, for bending the body duct tissue, represented by arrow 48, to narrow the angle with respect to the anchor assembly 12, and hence narrow the separation between the wall sections. The bending mechanism may, for example, be implemented by the steering and/or deflection capability of the respective assembly or assemblies.

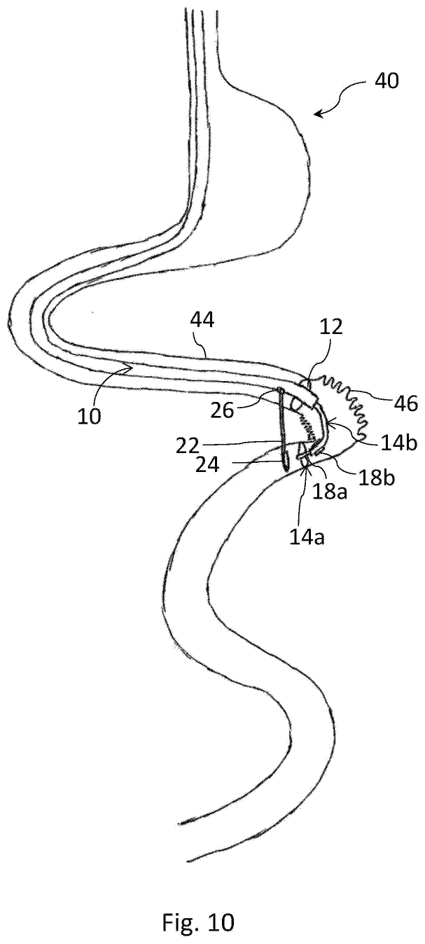

Referring to FIGS. 10 to 12 and 16e-g, a second example of lateral movement device is illustrated in the form of the second pulling assembly comprising the spanning element 22 extendable from the anchor assembly 12. The spanning element 22 is configured to penetrate through the duct wall tissue as the spanning element 22 extends from the anchor assembly 12, and passes outside the body duct to span the distance between the two sections of wall tissue. The spanning element 22 may optionally be steerable to enable fine adjustment of the position at which the spanning element 22 contacts the opposing tissue wall, and to avoid interfering with other adjacent body tissues. By using the first pulling assembly first to contract the body duct lengthwise, and hence draw distant tissues closer together, and optionally the additional bending provided by deflecting the first pulling assembly, the spanning element 22 need span only a relatively short distance in order to connect to opposing tissue. Extension of the spanning element 22 to span across the tissue positions outside the duct needs only local guidance, for example, fluoroscopy or ultrasound imaging, because the distance is relatively short.

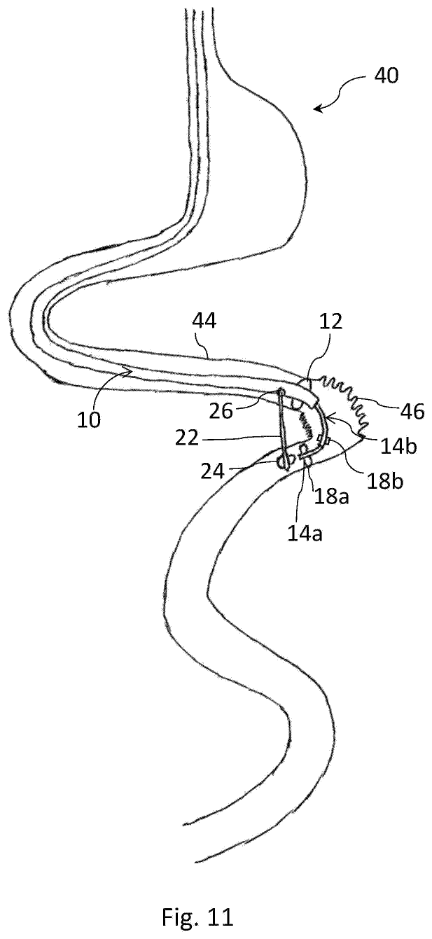

In the illustrated examples, the spanning element 22 penetrates the opposed tissue wall section, after which the distal shoulder 24 may be deployed (FIGS. 11 and 16f) to engage behind the tissue wall, and enable the tissue wall to be pulled by the spanning element 22. In an alternative embodiment, the spanning element 22 may carry a suction port (not shown) allowing the opposed tissue wall section to be engaged and pulled using negative pressure applied via a suitable conduit in the apparatus.

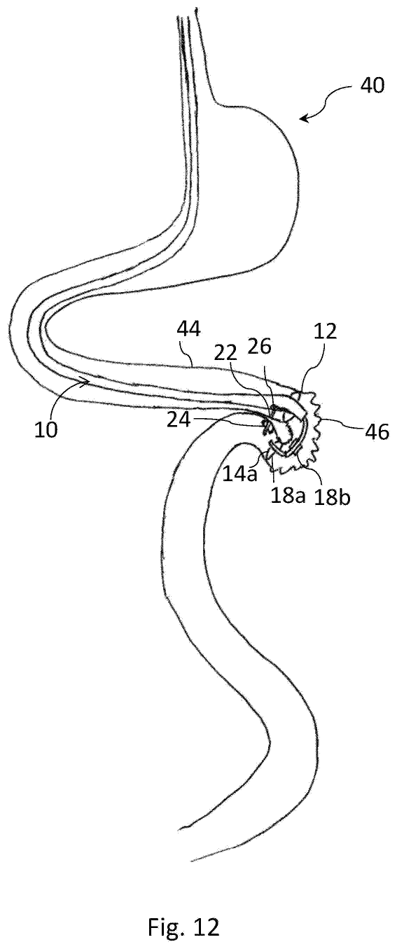

Referring to FIGS. 12 and 16g, retraction of the spanning element 22 towards the anchor assembly 12 pulls the two sections of body duct tissue into wall-to-wall engagement, for example, exterior-wall to exterior-wall engagement, thereby creating endoluminally a target site for an anastomosis. During such movement, one or both of the inflatable balloons 18a and 18b of the inners assemblies 14 and 14b may be deflated so as not to obstruct bending of the body duct into wall-to-wall engagement (FIG. 12). Additionally or alternatively, one or both of the inner assemblies may be retracted and removed from the tissue region 46 (FIGS. 16f and 16g).

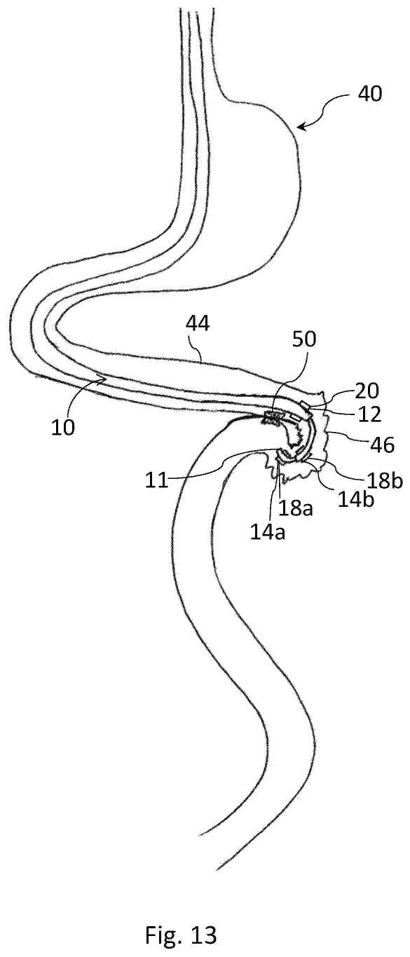

Referring to FIG. 13, a deployment device and/or tool 50 may then be used to create an anastomosis at the target site. In the illustrated example, a device 50 may be deployed by parachuting or guiding over the spanning element 22. The device 50 may comprise an anastomosis stent for forming a stented anastomosis, and/or a magnetic device for forming a magnetic compression anastomosis. The device 50 may comprise a single part, or multiple parts functioning collectively. Respective parts or portions of device 50, e.g. magnetically attracted parts or portions, may be deployed on opposite sides of the apposed tissue wall sections, to sandwich the tissue wall sections together, optionally coupled by structure passing through the body duct tissue walls, optionally not directly coupled together. The device 50 may be a permanent implant or a temporary implant. Additionally or alternatively, one or more tools (not shown) may be deployed for creating the anastomosis, for example a tissue cutting tool, and/or a tissue fixation tool. Tissue fixation may be achieved by sutures or staples or by other tissue fixation elements. The device or toll 50 may be advanced through a working channel of the apparatus 10 that communicates with the aperture 26 in the anchor assembly 12, or it may be loaded into an accommodation region (not shown) of the apparatus near its distal end region, ready for deployment and/or use.

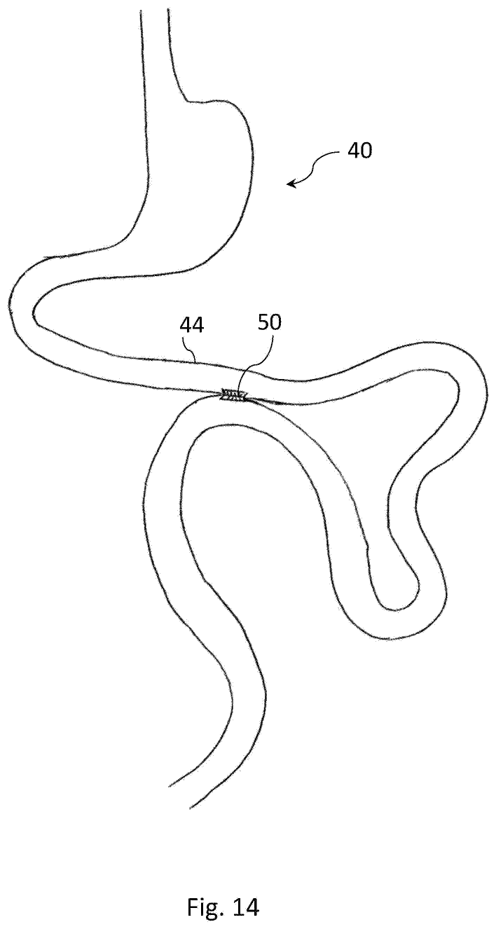

Referring to FIG. 14, withdrawal of the apparatus 10 leaves the created anastomosis or anastomosis device 50 in place providing a bypass in the body duct.

It will be appreciated that the above technique can facilitate a substantially endoluminal technique for anastomosis creation.

Body duct tissues deep within the anatomy, for example, deep in the small intestine, can be accessed and manipulated to create a target site for the anastomosis even if the tissues are not already in close proximity with each other. The technique of contracting body duct tissues lengthwise from within the body duct by pulling distant tissue towards the anchor assembly 12, followed by laterally moving spaced apart tissues towards and/or into wall-to-wall engagement, greatly expands the possibilities for minimally invasive anastomosis creation by practitioners.

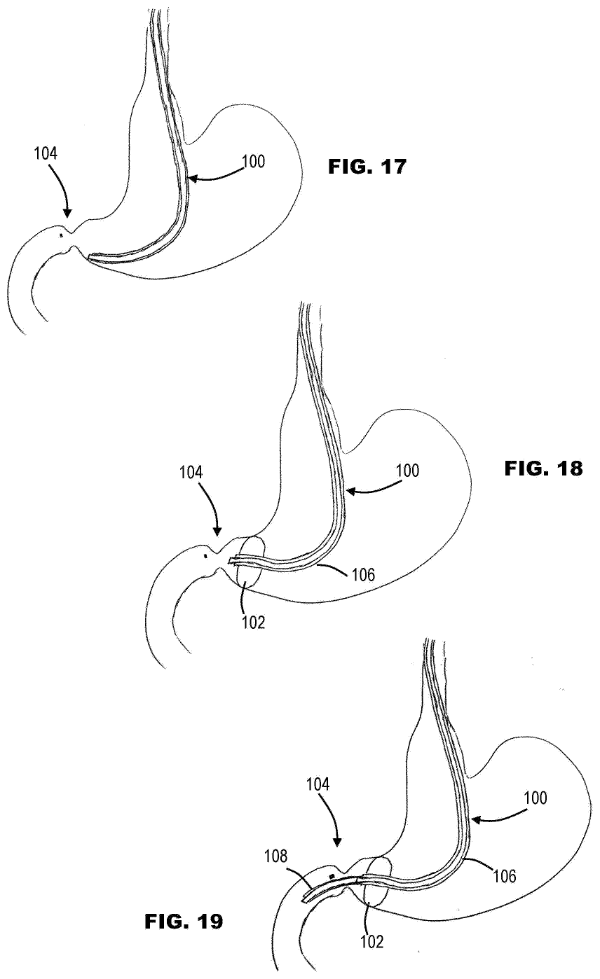

FIGS. 17-19 illustrate a further example in which an inflatable balloon 102 of a catheter 100 is used to assist endoluminal navigation of the catheter 100 (or a component) for traversing a restriction in the gastro-intestinal tract. In the illustrated example, the restriction is the pylorus 104. The catheter 100 may optionally be or include the apparatus 10 described above, or it may be a different apparatus. For example, the catheter 100 may be a delivery catheter configured for delivering an implant at least partly through the pylorus. The implant may comprise any of: a duodenal sleeve; an anchor for a duodenal sleeve; a duodenal anchor for a sleeve; a gastric anchor for a sleeve; a transpyloric anchor for a sleeve.

Referring to FIG. 17, the catheter 100 is introduced into the patient's stomach through the patient's mouth, and advanced towards the antrum. As illustrated in FIG. 17, and especially in the case of an unguided catheter, the catheter 100 may tend to adopt a shape with minimal curvature, such that the tip of the catheter is not optimally aligned with the through-passage of the pylorus. In some cases, such a catheter may tend to lodge in the pyloric antrum, and/or it may be awkward to traverse the pylorus 104.

Referring to FIG. 18, in the present example, an elongate tubular member 106 of the catheter 100 comprises an inflatable balloon 102, for example, near or at the tip. Inflation of the balloon 102 displaces the tip away from the tissue wall, and enhances alignment between the catheter 100 and the pylorus 104.

Referring to FIG. 19, following inflation of the balloon 102, a second elongate member 108 is slidable within the first elongate member, to extend beyond the tip, and traverse the pylorus 104 from the position displaced away from the tissue wall by the inflated balloon.

The balloon 102 may optionally be one of the inflatable balloons described in the preceding example, used here to facilitate navigation of the apparatus towards a target site beyond the pylorus.

It will be appreciated that use of the balloon 102 can facilitate navigation through a duct restriction, for example, the pylorus, even in the case of an unguided catheter 100.

It is emphasized that the above description merely refers to a non-limiting example of the invention, and that many modifications and equivalents may be used within the scope and/or principles of the invention.

1.-25. (canceled)

26. An apparatus insertable into a body duct and operable to manipulate body duct tissue to create a target site for an anastomosis by bringing sections of the body duct tissue into close relation with each other, the apparatus comprising:
an anchor assembly including an anchor for anchoring at a selected position within the body duct; and
at least a first pulling assembly configured for contracting a portion of the body duct in a lengthwise direction from within the body duct, by pulling distant tissue towards the anchor assembly, the first pulling assembly comprising at least one elongate member that is extendable and retractable for movement within the body duct, and at least one distal balloon inflatable for frictionally engaging an interior surface of the body duct to enable the frictionally engaged tissue to be pulled towards the anchor assembly.

27. The apparatus according to claim 26, wherein the first pulling assembly comprises:
a first elongate member carrying a first distal inflatable balloon; and
a second elongate member carrying a second distal inflatable balloon.

28. The apparatus according to claim 26, wherein the at least one elongate member of the first pulling assembly is at least partly deflectable and/or steerable for navigating when extending within the body duct.

29. The apparatus according to claim 26, wherein the deployable anchor of the anchor assembly comprises an inflatable balloon.

30. The apparatus according to claim 26, further comprising a lateral movement device configured for moving first and second sections of the body duct tissue that are spaced lengthwise along the body duct, laterally towards and/or into wall-to-wall engagement with each other.

31. The apparatus according to claim 29, wherein the lateral movement device comprises a second pulling assembly configured for spanning between the first and second sections by passing outside the body duct, and for pulling the spanned sections towards wall-to-wall engagement with each other.

32. The apparatus according to claim 31, wherein the second pulling assembly comprises an extendable and retractable spanning element for at least one of: passing through the tissue wall section; and spanning the distance outside the body duct between tissue wall sections.

33. The apparatus according to claim 32, wherein the spanning element is deflectable and/or steerable for navigating when extending to span between the first and second sections.

34. The apparatus according to claim 31, wherein the second pulling assembly further comprises a connecting device for connecting with a tissue wall section in such a manner that enables that tissue wall section to be pulled, the connecting device comprising at least one of: (i) a deployable distal shoulder for engaging behind body duct tissue at one of the sections for enabling the tissue wall section to be pulled; and/or (ii) a suction port for connecting with tissue by application of negative pressure that sucks the tissue against the suction port.

35. The apparatus according to claim 34, wherein the deployable shoulder comprises an element selected from one or more of: an inflatable balloon; a stent; a mechanical structure; and a self-expanding element.

36. The apparatus according to claim 26, further comprising a deployment device for deploying an anastomosis device.

37. The apparatus according to claim 26, further comprising a deployment device for deploying a tool for use in anastomosis creation.

38. An apparatus insertable into a body duct and operable to manipulate body duct tissue to create a target site for an anastomosis by bringing sections of the body duct tissue into close relation with each other, the apparatus comprising:
an anchor assembly including an anchor for anchoring at a selected position within the body duct;
at least a first pulling assembly configured for contracting a portion of the body duct in a lengthwise direction from within the body duct, by pulling distant tissue towards the anchor assembly;
the apparatus further comprising at least one of:
a lateral movement device configured for moving first and second sections of the body duct tissue that are spaced lengthwise along the body duct, laterally towards and/or into wall-to-wall engagement with each other;
and/or a deployment device for deploying one or more tools and/or devices for creating an anastomosis.

39. The apparatus according to claim 38, wherein the lateral movement device comprises a second pulling assembly configured for spanning positions along the body duct by passing outside the body duct, and for pulling the spanned positions towards wall-to-wall engagement with each other.

40. A method of endoscopically manipulating body duct tissue to create a target site for an anastomosis by bringing sections of body duct tissue into close relation with each other, using an apparatus insertable endoluminally into the body duct, the method comprising:
deploying an anchor of the apparatus to anchor the apparatus at a selected position within the body duct;
operating a first pulling assembly of the apparatus to contract a portion of the body duct in a lengthwise direction from within the body duct, by pulling distant tissue towards the anchor assembly;
the method further comprising at least one of the following steps:
operating a lateral movement device of the apparatus to move first and second sections of the body duct tissue that are spaced lengthwise along the body duct, laterally towards and/or into wall-to-wall engagement with each other; and/or
operating a deployment device to deploy one or more tools and/or devices for creating an anastomosis.

41. The method according to claim 40, wherein operating the first pulling assembly comprises contracting a portion of the body duct in a lengthwise direction such that the first and second sections of body duct tissue move from a distant relation to a closer relation in the lengthwise direction; and.

42. A method of endoscopically navigating within the gastro-intestinal tract of a patient, the method comprising:
advancing a first elongate tubular member within the gastro-intestinal tract to position a first portion of the first member in the region of a restriction of the tract to be traversed;
inflating a balloon of the first elongate tubular member to displace the first portion of the first elongate tubular member away from the wall of the tract; and
advancing a second elongate member within the first elongate member, to pass through the restriction of the tract from a position spaced from the tissue wall by the balloon of the first elongate tubular member.

43. The method according to claim 42, wherein the first portion of the first elongate member is at or near a tip of the first elongate member.

44. The method according to claim 42, wherein the restriction of the gastro-intestinal tract is a pylorus.

45. The method according to claim 42, further comprising a step of deploying an implant into the gastro-intestinal tract, wherein the implant comprises at least one selected from: a duodenal sleeve; a duodenal anchor for a duodenal sleeve; a trans-pyloric anchor for a duodenal sleeve; a gastric anchor for a duodenal sleeve.
